(19) United States
(12) Patent Application Publication
(10) Pub. No.: US 2011/0098367 A1
(43) Pub. Date: Apr. 28, 2011

(54) METHOD & APPARATUS FOR PROCESSING FISCHER-TROPSCH OFF-GAS

(76) Inventors: Gerrit Jan Barend ASSINK, Amsterdam (NL); Eva Marfilia Van Dorst, Amsterdam (NL); Ulrich Markus Hinrich Hennings, Amsterdam (NL)

(21) Appl. No.: 12/907,818

(22) Filed: Oct. 19, 2010

(30) Foreign Application Priority Data
Oct. 21, 2009 (EP) 09173662.9

(51) Int. Cl. C07C 1/00; B01D 53/62

(52) U.S. Cl. 518/705; 422/171

(57) ABSTRACT

This invention concerns methods and apparatus for processing Fischer-Tropsch off-gas comprising the following steps: a) subjecting Fischer-Tropsch off-gas to at least a water gas shift reaction and partial CO₂ removal resulting in a Fischer-Tropsch off-gas with significantly reduced levels of carbon dioxide and a CO₂ rich stream; and optionally b) subjecting part of the carbon dioxide depleted Fischer-Tropsch off-gas to synthesis gas manufacturing; and c) using another part of the carbon dioxide depleted Fischer-Tropsch off-gas for generating energy.

This application claims the benefit of European Application No. 09173662.9 filed Oct. 21, 2009, which is incorporated herein by reference.

### FIELD OF THE INVENTION

The present invention relates to a process for processing Fischer-Tropsch off-gas.

### BACKGROUND OF THE INVENTION

The Fischer-Tropsch process can be used for the conversion of hydrocarbonaceous feed stocks into normally liquid and/or solid hydrocarbons (i.e. measured at 0° C., 1 bar). The feed stock (e.g. natural gas, associated gas, coal-bed methane, residual oil fractions, biomass and/or coal) is converted in a first step into a mixture of hydrogen and carbon monoxide. This mixture is often referred to as synthesis gas or syngas. The synthesis gas is fed into a reactor where it is converted over a suitable catalyst at elevated temperature and pressure into paraffinic compounds ranging from methane to high molecular weight molecules comprising up to 200 carbon atoms, or, under particular circumstances, even more.

The hydrocarbon products manufactured in the Fischer-Tropsch process are processed into different fractions, for example a liquid hydrocarbon stream comprising mainly C₅+ hydrocarbons, and a gaseous hydrocarbon stream which comprises carbon monoxide, uncoverted methane, and lower hydrocarbons. The gaseous hydrocarbon stream is often referred to as Fischer-Tropsch off-gas. After separation of the liquid hydrocarbons, part of the Fischer-Tropsch off-gas can be recycled to the syngas manufacturing and part of the off-gas can be used as fuel. Usually, the recycle stream is subjected to carbon dioxide removal before being recycled to the syngas manufacturing.

WO03/104355 discloses a process for the conversion of hydrocarbonaceous feed by partial oxidation using an oxygen containing gas into synthesis gas. Subsequently, this synthesis gas is catalytically converted into hydrocarbons using a Fischer-Tropsch catalyst. The Fischer-Tropsch product is separated into a hydrocarbon product stream containing a relatively large amount of hydrocarbons in the C10-C14 range and a Fischer-Tropsch off-gas. One part of the Fischer-Tropsch off-gas is subjected to carbon dioxide removal and another part is used as fuel for generating energy. The carbon dioxide depleted Fischer-Tropsch off-gas is recycled to the partial oxidation process.

EP1004561 describes a process for producing liquid hydrocarbons comprising the steps of manufacturing syngas by partial oxidation of hydrocarbonaceous feeds at elevated temperature and pressure, catalytically converting the syngas into, in al., liquid hydrocarbons and Fischer-Tropsch off-gas, and expanding and/or combusting at least part of the Fischer-Tropsch off-gas to provide power for compressing the hydrocarbonaceous feed used in the syngas manufacture.

US2002/120017 describes the production of power, liquid hydrocarbons and carbon dioxide from a hydrocarbonaceous feed using partial oxidation (PDX) and Fischer-Tropsch reactors. The off-gas (referred to as tail gas) obtained by the described method may be subjected to steps of separation of carbon dioxide and hydrogen, the resultant product of which can then be returned to a partial oxidation reactor or sent to a gas turbine for power production.

WO03/035590 describes processes for handling of Fischer-Tropsch tail gas (referred to herein as off-gas). In particular, this document describes processing of tail gas to remove carbon dioxide and then splitting the ‘sweetened’ tail gas into streams for recycling into the Fischer-Tropsch reactor, sending to a power block (turbine) or recycling into a PDX reactor for additional syn gas production.

A disadvantage of the prior art processes described is that the part of the Fischer-Tropsch off-gas used for generating energy comprises a large amount of CO₂. Combustion of the CO₂ comprising Fischer-Tropsch off-gas in power generation will lead to CO₂ emission. The gas obtained after combustion is typically referred to as flue gas. Removal of CO₂ from the flue gas is too expensive, e.g. chemical absorption requires large scale equipment and energy consumption. In addition, the prior art does not consider the implications of combustion of other carbon containing compounds present in the off-gas and the effects on the levels of carbon dioxide in the flue gas.

### SUMMARY OF THE INVENTION

A first aspect of the invention provides a method for processing a Fischer-Tropsch off-gas comprising the following steps:

i) conversion of a (gaseous) hydrocarbonaceous feed to obtain synthesis gas;
ii) catalytic conversion of the synthesis gas obtained in step i) using a Fischer-Tropsch catalyst into a Fischer-Tropsch product;
iii) separating the Fischer-Tropsch product of step ii) into at least one hydrocarbon product stream and a Fischer-Tropsch off-gas;
iv) subjecting the Fischer-Tropsch off-gas to a water gas shift reaction resulting in a carbon monoxide depleted off-gas; and
v) subjecting the carbon monoxide depleted off-gas of step iv) to partial carbon dioxide removal resulting in a carbon dioxide depleted Fischer-Tropsch off-gas and a carbon dioxide rich stream.

In one embodiment of the invention the Fischer-Tropsch off-gas of step iii) is subjected to steam methane reforming reaction prior to the water gas shift reaction of step iv).

Optionally, in step v) part of the carbon dioxide depleted Fischer-Tropsch off-gas may be recycled to the hydrocarbonaceous feed for conversion to synthesis gas in step i). Alternatively, or in addition, in step v) part of the carbon dioxide depleted Fischer-Tropsch off-gas may be recycled to the synthesis gas feed obtained from step i) for catalytic conversion using a Fischer-Tropsch catalyst into a Fischer-Tropsch product. Further, it is also an option in step v) for part of the carbon dioxide depleted Fischer-Tropsch off-gas is used for generating energy.

In a specific embodiment of the invention the Fischer-Tropsch off-gas is subjected to the carbon dioxide removal at a temperature in the range of between about 40° C. and about 100° C. and at a pressure in the range of between about 40 bar to about 80 bar. In a further embodiment, during step v) at least 70 vol. % of carbon dioxide is removed from the Fischer-Tropsch off-gas, calculated on the total amount of carbon dioxide in the Fischer-Tropsch off-gas. Typically, the carbon dioxide rich stream is stored or re-used.

In one embodiment of the invention the water gas shift reaction of step iv) occurs at a pressure in the range of between about 10 bar and 30 bar and a temperature in the range of between about 150° C. to 250° C.

In a further embodiment of the invention the steam methane reforming reaction occurs at a pressure in the range of between about 25 bar and about 30 bar and a temperature in the range of between about 820° C. to about 850° C.

A second aspect of the invention provides for a processed Fischer-Tropsch off-gas composition obtainable according to the process as described herein, characterised in that the off-gas is enriched for hydrogen and depleted of carbon dioxide and carbon monoxide.

A third aspect of the invention provides for a processed Fischer-Tropsch off-gas composition obtainable according to the process as described herein, characterised in that the off-gas is enriched for hydrogen and depleted of carbon dioxide, carbon monoxide and methane.

A fourth aspect of the invention provides an apparatus for the production of liquid hydrocarbons comprising:

a) a partial oxidation (PDX) reactor, for conducting partial oxidation of a hydrocabonaceous feedstock so as to produce a synthesis gas;
b) a Fischer-Tropsch reactor, wherein the Fischer-Tropsch is in fluid communication with the PDX reactor of a) and comprises Fischer-Tropsch catalyst, the Fischer-Tropsch reactor being adapted to effect conversion of the synthesis gas into a Fischer-Tropsch product;
c) a separator, wherein the separator is adapted to receive the Fischer-Tropsch product from the Fischer-Tropsch reactor of b), the separator being for separating the Fischer-Tropsch product into a liquid hydrocarbon product stream and an off-gas stream;
d) a water gas shift reactor, wherein the water gas shift reactor is adapted to receive the off-gas stream from the separator of c), the water gas shift reactor being for performance of a water gas shift reaction on the off-gas so as to generate a carbon monoxide depleted off-gas stream; and
e) a carbon capture plant, wherein the carbon capture plant is adapted to receive the carbon monoxide depleted off-gas stream from the water gas shift reactor of d), wherein the carbon capture plant is for removal and sequestration of carbon dioxide present within carbon monoxide depleted off-gas stream, thereby generating a carbon dioxide depleted off-gas product stream and a carbon dioxide enriched product stream.

In a specific embodiment the apparatus further comprises:

f) a power generation plant, wherein the power generation plant is adapted to receive at least a portion of the carbon dioxide depleted off-gas product stream from the carbon capture plant of e).

A fifth aspect of the invention provides an apparatus for the production of liquid hydrocarbons comprising:

a) a partial oxidation (PDX) reactor, for conducting partial oxidation of a hydrocabonaceous feedstock so as to produce a synthesis gas;
b) a Fischer-Tropsch reactor, wherein the Fischer-Tropsch is in fluid communication with the PDX reactor of a) and comprises Fischer-Tropsch catalyst, the Fischer-Tropsch reactor being adapted to effect conversion of the synthesis gas into a Fischer-Tropsch product;
c) a separator, wherein the separator is adapted to receive the Fischer-Tropsch product from the Fischer-Tropsch reactor of b), the separator being for separating the Fischer-Tropsch product into a liquid hydrocarbon product stream and an off-gas stream;
d) a steam methane reformer, wherein the steam methane reformer is adapted to receive the off-gas stream from the separator of c), steam methane reformer being for the purpose of subjecting the off-gas stream to steam methane reforming so as to generate a methane depleted off-gas stream;
e) a water gas shift reactor, wherein the water gas shift reactor is adapted to receive the methane depleted off-gas stream from the steam methane reformer of d), the water gas shift reactor being for performance of a water gas shift reaction on the methane depleted off-gas so as to generate a carbon monoxide and methane depleted off-gas stream; and
f) a carbon capture plant, wherein the carbon capture plant is adapted to receive the carbon monoxide and methane depleted off-gas stream from the water gas shift reactor of e), wherein the carbon capture plant is for removal and sequestration of carbon dioxide present within carbon monoxide and methane depleted off-gas stream, thereby generating a carbon dioxide depleted off-gas product stream and a carbon dioxide enriched product stream.

In a specific embodiment of the invention the apparatus further comprises:

g) a power generation plant, wherein the power generation plant is adapted to receive at least a portion of the carbon dioxide depleted off-gas product stream from the carbon capture plant of f).

Optionally at least a portion of the carbon dioxide depleted off-gas product stream may be combined with the hydrocarbonaceous feedstock prior to step a) as described above. Alternatively, or in addition, at least a portion of the carbon dioxide depleted off-gas product stream may be combined with the synthesis gas obtained from the PDX reactor of a) prior to step b).

An advantage of the process according to the invention is that not only the fraction that is subjected to syngas manufacturing is a carbon dioxide depleted Fischer-Tropsch off-gas but also the fraction that will be combusted. In this way CO₂, methane and CO are removed from the Fischer-Tropsch off-gas at high pressure before this Fischer-Tropsch off-gas is combusted for generating energy instead of using the difficult and expensive process for removal of CO₂ from the flue gas. The methods and apparatus of the present invention are able to reduce carbon dioxide levels in flue gas by between 20 and 50 vol. %, typically around 30 vol. % when compared to off-gas processing using only a carbon capture unit.

The invention is further illustrated in the accompanying drawings.

### DRAWINGS

FIG. 1 shows a flow chart of a process according to the prior art.

FIG. 2 shows a flow chart of a process according to a first embodiment of the invention.

FIG. 3 shows a flow chart of a process according to a second embodiment of the invention.

### DETAILED DESCRIPTION OF THE INVENTION

The present invention relates to processing of off-gas obtained from a Fischer-Tropsch reactor in order to reduce overall carbon load. When removed from a Fischer-Tropsch reactor, the Fischer-Tropsch off-gas is generally at a temperature in the range of 40-100° C., preferably in the range of 50-70° C. and at a pressure of 40-80 bar, preferably in the range of 50-70 bar.

Fischer-Tropsch off-gas is typically produced by a Fischer-Tropsch hydrocarbon synthesis process comprising the steps of:

i) conversion of a (gaseous) hydrocarbonaceous feed to obtain synthesis gas (syngas);
ii) catalytic conversion of the synthesis gas obtained in step i) using a Fischer-Tropsch catalyst into a Fischer-Tropsch product; and
iii) separating the Fischer-Tropsch product of step ii) into at least one hydrocarbon product stream and a Fischer-Tropsch off-gas.

Suitably, syngas production methods include steam reforming of natural gas or liquid hydrocarbons and gasification of coal. Methods to convert (gaseous) hydrocarbonaceous feed into syngas include adiabatic oxidative reforming, autothermal reforming and partial oxidation. Preferably, hydrocarbonaceous feed is converted to syngas by partial oxidation at elevated temperature and pressure using an oxygen containing gas. Partial oxidation can take place according to various established processes. Catalytic as well as non-catalytic processes may be used. These processes include the Shell Gasification Process. A comprehensive survey of this process can be found in the Oil and Gas Journal, Sep. 6, 1971, pp 86-90.

The H₂/CO ratio of the syngas is suitably between 1.5 and 2.3, preferably between 1.8 and 2.1. The catalysts used for the catalytic conversion of the mixture comprising hydrogen and carbon monoxide into hydrocarbons are known in the art and are usually referred to as Fischer-Tropsch catalysts. Preferably, the catalysts for use in the Fischer-Tropsch hydrocarbon synthesis process comprise cobalt as the catalytically active component. The catalytically active component is preferably supported on a porous carrier, e.g. silica or titania. If desired, the Fischer-Tropsch catalyst may also comprise one or more metals or metal oxides as promoters. Typically, the catalytic conversion may be effected at a temperature in the range of 150 to 350° C., preferably from 180 to 270° C. Typical total pressures for the catalytic conversion process are in the range of from 1 to 200 bar absolute, more preferably from 10 to 70 bar absolute.

Generally, the Fischer-Tropsch hydrocarbon product stream is separated from the Fischer-Tropsch off-gas by a gas/liquid separator.

The Fischer-Tropsch off-gas may comprise gaseous hydrocarbons, nitrogen, unconverted methane, unconverted carbon monoxide, carbon dioxide, hydrogen and water. The gaseous hydrocarbons are suitably C₁-C₅ hydrocarbons, preferably C₁-C₄ hydrocarbons, more preferably C₁-C₃ hydrocarbons. These hydrocarbons, or mixtures thereof, are gaseous at temperatures of 5-30° C. (1 bar), especially at 20° C. (1 bar). Further, oxygenated compounds, e.g. methanol, dimethylether, may be present.

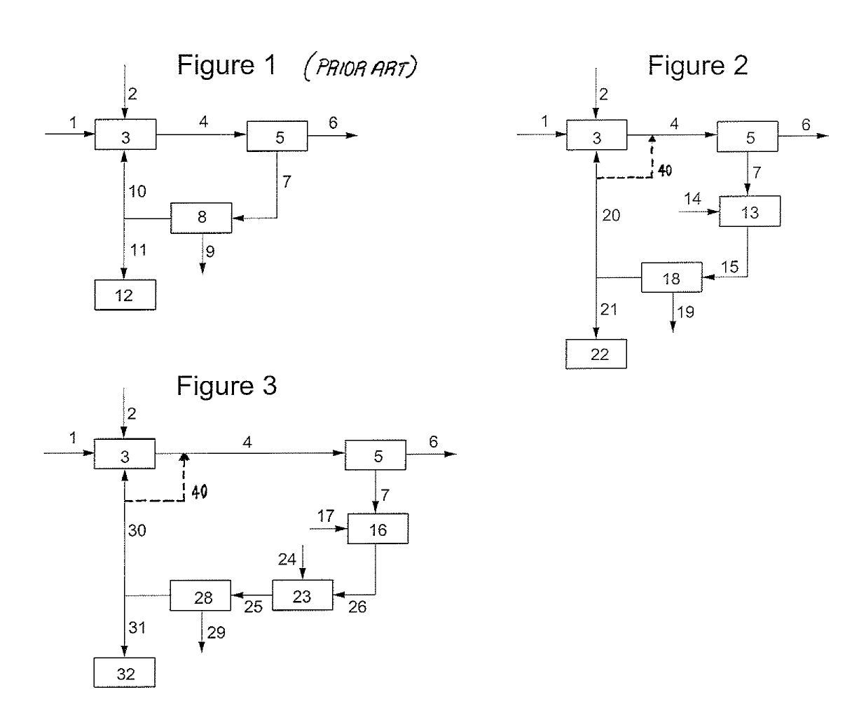

It is known in the art to subject the Fischer-Tropsch off-gas to partial CO₂ removal resulting in a carbon dioxide depleted Fischer-Tropsch off-gas (see FIG. 1).

Typically conventional prior art processes for processing Fischer-Tropsch off-gas, comprise the following steps:

i) conversion of a (gaseous) hydrocarbonaceous feed to obtain synthesis gas;
ii) catalytic conversion of the synthesis gas obtained in step i) using a Fischer-Tropsch catalyst into a Fischer-Tropsch product;
iii) separating the Fischer-Tropsch product of step ii) into at least one hydrocarbon product stream and a Fischer-Tropsch off-gas;
iv) subjecting Fischer-Tropsch off-gas to partial CO₂ removal resulting in a carbon dioxide depleted Fischer-Tropsch off-gas;
v) subjecting part of the carbon dioxide depleted Fischer-Tropsch off-gas to synthesis gas manufacturing; and
vi) using another part of the carbon dioxide depleted Fischer-Tropsch off-gas for generating energy.

The primary draw back with conventional processes for reducing carbon levels in the off-gas is that they concentrate solely on removal of the carbon dioxide generated during the Fischer-Tropsch reaction and do not consider other carbon containing components within the off-gas mixture, such as methane and carbon monoxide. Further, prior art methods do not seek to maximise the concentration of carbon dioxide in the off-gas by converting other carbon containing components (such as carbon monoxide or methane) into carbon dioxide. According to the present invention, maximising the carbon dioxide concentration in the off-gas stream prior to the carbon capture step greatly increases the efficiency of carbon dioxide recovery and leads to production of a high purity carbon dioxide rich stream which represents a valuable product of the process in its own right.

According to the present invention CO₂ can be removed at the temperature and pressure at which the Fischer-Tropsch off-gas is generally at, when removed from a Fischer-Tropsch reactor, thus at a temperature in the range of 40-100° C., preferably in the range of 50-70° C. and at a pressure of 40-80 bar, preferably in the range of 50-70 bar.

For the removal of carbon dioxide any suitable conventional process may be used, for instance adsorption processes using amines, especially in combination with a physical solvent, such as the ADIP process or the SULFINOL process as described in inter alia GB 1,131,989; GB 965,358; GB 957260; and GB 972,140. Carbon dioxide removal is often referred to as carbon capture, which is usually part of carbon capture and storage processes.

The CO₂ rich stream may be stored or re-used. CO₂ storage may for example, include gaseous storage in various deep geological formations (including saline formations and exhausted gas fields), liquid storage in the ocean, and/or solid storage by reaction of CO₂ with metal oxides to produce stable carbonates. Carbon dioxide storage is often referred to as CO₂ sequestration, which is usually part of carbon capture and storage processes (CCS). Additionally or alternatively, CO₂ may be re-used for enhanced oil recovery and/or for plant growth and production within a greenhouse environment and/or for pelleting and using in industrial cooling applications.

Preferably, at least 70 vol. %, more preferably between 60 and 80 vol. %, even more preferably at least 90 vol. % of CO₂ is removed from the Fischer-Tropsch off-gas, calculated on the total amount of CO₂ in the Fischer-Tropsch off-gas. Part of the CO₂ depleted Fischer-Tropsch off-gas is subjected to syngas manufacturing, in which process the hydrocarbonaceous feed is converted into synthesis gas. Preferably, between 50 and 90 vol. %, more preferably between 60 and 80 vol. %, of the CO₂ depleted Fischer-Tropsch off-gas is subjected to syngas manufacturing.

According to an embodiment of the present invention, part of the CO₂ depleted Fischer-Tropsch off-gas is recycled to syngas manufacturing.

Preferably, between 50 and 90 vol. %, more preferably between 60 and 80 vol. %, of the CO₂ depleted Fischer-Tropsch off-gas is recycled to syngas manufacturing.

Part of the CO₂ depleted Fischer-Tropsch off-gas is used as fuel for generating energy. Preferably, between 5 and 50 vol. %, more preferably between 10 and 40 vol. %, of the CO₂ depleted Fischer-Tropsch off-gas is used as fuel.

In this way, not only the part of the Fischer-Tropsch off-gas that is recycled to the syngas manufacture is treated for CO₂ removal but also the part which is used for generating energy. The flue gas obtained after combustion of carbon dioxide depleted Fischer-Tropsch off-gas will contain less CO₂ in comparison with flue gas obtained after combustion of carbon dioxide rich Fischer-Tropsch off-gas.

According to one aspect of the present invention, the Fischer-Tropsch off-gas is subjected to a water gas shift reaction before being subjected to the partial CO₂ removal. The Fischer-Tropsch off-gas is mixed with steam, and CO present in the Fischer-Tropsch off-gas is reacted to form H₂ and CO₂. Preferably, the off-gas is mixed with steam at a pressure of 10 to 30 bar and a temperature in the range of 150° C. to 250° C. This reaction can be performed with any suitable catalyst, for example a CuZnO catalyst. In this way, the Fischer-Tropsch off-gas will be depleted from CO. At the same time, the off-gas is enriched for H₂. Hereafter, the CO depleted Fischer-Tropsch off-gas is subjected to partial CO₂ removal. Combustion of a CO and CO₂ depleted Fischer-Tropsch off-gas will lead to a flue gas containing even less CO₂ in comparison with flue gas obtained after combustion of only a carbon dioxide depleted Fischer-Tropsch off-gas as provided by the prior art.

In a further aspect of the invention, the Fischer-Tropsch off-gas is subjected to steam methane reforming and subsequently to a water gas shift reaction before being subjected to the partial CO₂ removal. In steam methane reforming, the Fischer-Tropsch off-gas is mixed with steam, and the methane present in the Fischer-Tropsch off-gas is reacted to H₂ and CO₂. Preferably, the off-gas is mixed with steam at a pressure of 25 to 30 bar and at a temperature in the range of 820° C. to 850° C. This reaction can be performed with any suitable catalyst, for example a Ni-doped alumina catalyst. In this way, the Fischer-Tropsch off-gas will be depleted from methane. The methane depleted Fischer-Tropsch off-gas is in a next step subjected to a water gas shift reaction. Then a methane and CO depleted Fischer-Tropsch off-gas is obtained. Hereafter, the methane and CO depleted Fischer-Tropsch off-gas is subjected to partial CO₂ removal. Combustion of a methane, CO and CO₂ depleted Fischer-Tropsch off-gas will lead to a flue gas containing even less CO₂ in comparison with flue gas obtained after combustion of a CO₂ depleted, or even a CO₂ and CO depleted, Fischer-Tropsch off-gas.

Various embodiments of the process and apparatus according to the invention will be illustrated below with reference to the attached figures. It is noted that the present invention should not be considered limited thereto or thereby.

FIG. 1 illustrates the prior art process in which Fischer-Tropsch off-gas is immediately subjected to CO₂ depletion. In the process of FIG. 1, a gaseous hydrocarbonaceous feed (e.g. natural gas, associated gas, coal-bed methane, residual oil fractions, biomass and/or coal) is provided through line 1 and oxygen containing gas is provided through line 2 to a syngas manufacturing unit 3. In the syngas manufacturing unit 3, the gaseous hydrocarbonaceous feed is converted into synthesis gas, for example by partial oxidation. The effluent from the manufacturing unit 3 is fed through line 4 to a Heavy Paraffin Synthesis (HPS) unit 5. In unit 5, syngas is catalytically converted into a hydrocarbons using a Fischer-Tropsch catalyst. From unit 5 a C₅+ hydrocarbon comprising stream is separated and a Fischer-Tropsch off-gas is separated. The C₅+ hydrocarbon comprising stream is separated off through line 6. The Fischer-Tropsch off-gas is fed through line 7 to a Carbon Capture unit 8 in which CO₂ is removed from the Fischer-Tropsch off-gas.

A CO₂ rich stream is separated off through line 9. Suitably, the CO₂ is stored or re-used.

Part of the carbon dioxide depleted off-gas can be recycled through line 10 to the syngas manufacturing unit 3. Another part of the carbon dioxide depleted off-gas can be fed through line 11 to a furnace 12.

The flue gas obtained after combustion of carbon dioxide depleted Fischer-Tropsch off-gas will contain less CO₂ in comparison with flue gas obtained after combustion of carbon dioxide rich Fischer-Tropsch off-gas. However, overall carbon levels can still remain high due to the presence of CO and methane in the off-gas.

FIG. 2 illustrates a first embodiment of a process according to the invention. The same initial procedure is followed as in FIG. 1, until a separated Fischer-Tropsch off-gas from unit 5 is obtained. Hereafter, the Fischer-Tropsch off-gas is fed trough line 7 to a water gas shift reactor 13. Steam is provided through line 14 to reactor 13, in which the Fischer-Tropsch off-gas is mixed with steam, and the CO present in the Fischer-Tropsch off-gas is reacted to H₂ and CO₂.

The CO depleted Fischer-Tropsch off-gas is fed through line 15 to Carbon Capture unit 18, which CO₂ is removed from the Fischer-Tropsch off-gas. A CO₂ rich stream is separated off through line 19. Suitably, the CO₂ is stored or re-used.

Part of the carbon dioxide depleted Fischer-Tropsch off-gas is recycled through line 20 to the syngas manufacturing unit 3. Optionally, some or all of the carbon dioxide depleted off-gas may be diverted through line 40 and combined directly with the syngas in line 4. Another part of the carbon dioxide depleted off-gas is fed through line 21 to a furnace 22 for the purpose of power generation.

The flue gas obtained after combustion of CO₂ and CO depleted Fischer-Tropsch off-gas will contain considerably less CO₂ in comparison with flue gas obtained after combustion of CO₂ depleted Fischer-Tropsch off-gas. In addition, the efficiency of the carbon capture step is increased due to the higher concentration of carbon dioxide in the off-gas.

FIG. 3 illustrates a second embodiment of a process according to the invention. The same initial procedure was followed as in FIG. 1, until a separated Fischer-Tropsch off-gas from unit 5 is obtained. Hereafter, the Fischer-Tropsch off-gas is fed through line 7 to steam methane reformer 16. Steam is provided through line 17 to reformer 16, in which the Fischer-Tropsch off-gas is mixed with steam, and the methane present in the Fischer-Tropsch off-gas is reacted to H₂ and CO₂. The methane depleted Fischer-Tropsch off-gas is fed through line 26 to a water shift reactor 23. Steam is provided through line 24 to reactor 23, in which the Fischer-Tropsch off-gas is mixed with steam, and the CO present in the Fischer-Tropsch off-gas is reacted to H₂ and CO₂. The methane and CO depleted Fischer-Tropsch off-gas is fed through line 25 to Carbon Capture unit 28, in which carbon dioxide is removed from the Fischer-Tropsch off-gas. A carbon dioxide rich stream is separated off through line 29. Suitably, the carbon dioxide is stored or re-used.

Part of the carbon dioxide depleted off-gas is recycled through line 30 to the syngas manufacturing unit 3. Optionally, some or all of the carbon dioxide depleted off-gas may be diverted through line 40 and combined directly with the syngas in line 4. Another part of the carbon dioxide depleted off-gas is fed through line 31 to a furnace 32 for the purpose of power generation.

The flue gas obtained after combustion of a methane, CO₂ and CO depleted Fischer-Tropsch off-gas contains significantly less CO₂ in comparison with flue gas obtained after combustion of simply CO₂ depleted off-gas, or CO₂ and CO depleted Fischer-Tropsch off-gas. The process described in the embodiments of the invention allows for as much as a 50% reduction in CO₂ levels in the flue gas, typically around a 30% reduction. In this way, the method and apparatus of the invention provides for a significantly cleaner burning off-gas than was previously available in the art.

A further embodiment of the invention provides for inclusion of a hydrogen recovery unit (not shown) either before or after the carbon capture unit 18,28. Since the off-gas obtained following the water gas shift and optionally the steam methane reforming steps is significantly enriched for hydrogen, it may be desirable to separate the hydrogen from the off-gas in order to recycle it for use, for example, in balancing the H₂/CO ratio comprised within the syngas feed to the Fischer-Tropsch reactor 5. Hydrogen separation from the off-gas can be achieved via use of commercially available absorption membranes.

While the method and apparatus have been described in terms of what are presently considered to be the most practical and preferred embodiments, it is to be understood that the disclosure need not be limited to the disclosed embodiments. It is intended to cover various modifications, combinations and similar arrangements included within the spirit and scope of the claims, the scope of which should be accorded the broadest interpretation so as to encompass all such modifications and similar structures. The present disclosure includes any and all embodiments of the following claims.

It should also be understood that a variety of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes.

Any patents, publications, or other references mentioned in this application for patent are hereby incorporated by reference. In addition, as to each term used, it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans.

What is claimed is:

1. A method for processing a Fischer-Tropsch off-gas comprising the following steps:
i) conversion of a hydrocarbonaceous feed to obtain synthesis gas;
ii) catalytic conversion of the synthesis gas obtained in step i) using a Fischer-Tropsch catalyst into a Fischer-Tropsch product;
iii) separating the Fischer-Tropsch product of step ii) into at least one hydrocarbon product stream and a Fischer-Tropsch off-gas;
iv) subjecting the Fischer-Tropsch off-gas to a water gas shift reaction resulting in a carbon monoxide depleted off-gas; and
v) subjecting the carbon monoxide depleted off-gas of step iv) to partial carbon dioxide removal resulting in a carbon dioxide depleted Fischer-Tropsch off-gas and a carbon dioxide rich stream.

2. The method of claim 1, wherein the Fischer-Tropsch off-gas of step iii) is subjected to steam methane reforming reaction prior to the water gas shift reaction of step iv).

3. The method of claim 1 wherein in step v) part of the carbon dioxide depleted Fischer-Tropsch off-gas is recycled to the hydrocarbonaceous feed for conversion to synthesis gas in step i).

4. The method of claim 2 wherein in step v) part of the carbon dioxide depleted Fischer-Tropsch off-gas is recycled to the hydrocarbonaceous feed for conversion to synthesis gas in step i).

5. The method of claim 3 wherein in step v) part of the carbon dioxide depleted Fischer-Tropsch off-gas is recycled to the synthesis gas feed obtained from step i) for catalytic conversion using a Fischer-Tropsch catalyst into a Fischer-Tropsch product.

6. The method of claim 4 wherein in step v) part of the carbon dioxide depleted Fischer-Tropsch off-gas is recycled to the synthesis gas feed obtained from step i) for catalytic conversion using a Fischer-Tropsch catalyst into a Fischer-Tropsch product.

7. The method of claim 1 wherein in step v) part of the carbon dioxide depleted Fischer-Tropsch off-gas is used for generating energy.

8. The method of claim 1 wherein the carbon dioxide rich stream is stored or re-used.

9. An apparatus for the production of liquid hydrocarbons comprising:
a) a partial oxidation (PDX) reactor, for conducting partial oxidation of a hydrocabonaceous feedstock so as to produce a synthesis gas;
b) a Fischer-Tropsch reactor, wherein the Fischer-Tropsch is in fluid communication with the PDX reactor of a) and comprises Fischer-Tropsch catalyst, the Fischer-Tropsch reactor being adapted to effect conversion of the synthesis gas into a Fischer-Tropsch product;
c) a separator, wherein the separator is adapted to receive the Fischer-Tropsch product from the Fischer-Tropsch reactor of b), the separator being capable of separating the Fischer-Tropsch product into a liquid hydrocarbon product stream and an off-gas stream;
d) a water gas shift reactor, wherein the water gas shift reactor is adapted to receive the off-gas stream from the separator of c), the water gas shift reactor adapted for performance of a water gas shift reaction on the off-gas so as to generate a carbon monoxide depleted off-gas stream; and
e) a carbon capture plant, wherein the carbon capture plant is adapted to receive the carbon monoxide depleted off-gas stream from the water gas shift reactor of d), wherein the carbon capture plant is adapted for removal and sequestration of carbon dioxide present within carbon monoxide depleted off-gas stream, thereby generating a carbon dioxide depleted off-gas product stream and a carbon dioxide enriched product stream.

10. The apparatus of claim 9 wherein the apparatus further comprises:
f) a power generation plant, wherein the power generation plant is adapted to receive at least a portion of the carbon dioxide depleted off-gas product stream from the carbon capture plant of e).

11. An apparatus for the production of liquid hydrocarbons comprising:
a) a partial oxidation (PDX) reactor, for conducting partial oxidation of a hydrocabonaceous feedstock so as to produce a synthesis gas;
b) a Fischer-Tropsch reactor, wherein the Fischer-Tropsch is in fluid communication with the PDX reactor of a) and comprises Fischer-Tropsch catalyst, the Fischer-Tropsch reactor being adapted to effect conversion of the synthesis gas into a Fischer-Tropsch product;
c) a separator, wherein the separator is adapted to receive the Fischer-Tropsch product from the Fischer-Tropsch reactor of b), the separator being capable of separating the Fischer-Tropsch product into a liquid hydrocarbon product stream and an off-gas stream;
d) a steam methane reformer, wherein the steam methane reformer is adapted to receive the off-gas stream from the separator of c), steam methane reformer being adapted for subjecting the off-gas stream to steam methane reforming so as to generate a methane depleted off-gas stream;
e) a water gas shift reactor, wherein the water gas shift reactor is adapted to receive the methane depleted off-gas stream from the steam methane reformer of d), the water gas shift reactor being adapted for performance of a water gas shift reaction on the methane depleted off-gas so as to generate a carbon monoxide and methane depleted off-gas stream; and
f) a carbon capture plant, wherein the carbon capture plant is adapted to receive the carbon monoxide and methane depleted off-gas stream from the water gas shift reactor of e), wherein the carbon capture plant is adapted for removal and sequestration of carbon dioxide present within carbon monoxide and methane depleted off-gas stream, thereby generating a carbon dioxide depleted off-gas product stream and a carbon dioxide enriched product stream.

12. The apparatus of claim 11 wherein the apparatus further comprises:
g) a power generation plant, wherein the power generation plant is adapted to receive at least a portion of the carbon dioxide depleted off-gas product stream from the carbon capture plant of f).

13. The apparatus of claim 9 wherein at least a portion of the carbon dioxide depleted off-gas product stream is combined with the hydrocarbonaceous feedstock prior to step a) and wherein at least a portion of the carbon dioxide depleted off-gas product stream is combined with the synthesis gas obtained from the PDX reactor of a) prior to step b).

14. The apparatus of claim 11 wherein at least a portion of the carbon dioxide depleted off-gas product stream is combined with the hydrocarbonaceous feedstock prior to step a) and wherein at least a portion of the carbon dioxide depleted off-gas product stream is combined with the synthesis gas obtained from the PDX reactor of a) prior to step b).
